(19) United States
(12) Patent Application Publication
(10) Pub. No.: US 2022/0130079 A1
(43) Pub. Date: Apr. 28, 2022

(54) SYSTEMS AND METHODS FOR SIMULTANEOUS ATTENUATION CORRECTION, SCATTER CORRECTION, AND DE-NOISING OF LOW-DOSE PET IMAGES WITH A NEURAL NETWORK

(71) Applicant: Siemens Medical Solutions USA, Inc., Malvern, PA (US)

(72) Inventors: Jicun Hu, Knoxville, TN (US); Xiang Zhang, Knoxville, TN (US); William Whiteley, Knoxville, TN (US); Chuanyu Zhou, Knoxville, TN (US); Vladimir Panin, Knoxville, TN (US)

(21) Appl. No.: 16/949,277

(22) Filed: Oct. 23, 2020

(51) Int. Cl. G06T 11/00; G06T 5/00; A61B 6/03; A61B 6/00

(52) U.S. Cl. CPC G06T 11/005; G06T 11/006; G06T 5/002; G06T 2207/20084; A61B 6/5282; G06T 2207/10104; G06T 2207/20081; A61B 6/037

(57) ABSTRACT

An image reconstruction system generates de-noised, attenuation corrected, and scatter corrected images using AI processing. The system receives a low-dose PET image and applies a machine learning algorithm via a convolutional neural network to the low-dose PET image to generate an output image. The output image includes correction for scatter and attenuation associated with the image being low-dose. The system provides the output image to a computing device comprising a user interface.

### TECHNICAL FIELD

Aspects of the present disclosure relate in general to medical diagnostic systems and, more particularly, to training and using a neural network for reconstructing images from low-dose PET data.

### BACKGROUND

Nuclear imaging systems can employ various technologies to capture images. For example, some nuclear imaging systems employ positron emission tomography (PET) to capture images. PET is a nuclear medicine imaging technique that produces tomographic images representing the distribution of positron emitting isotopes within a body. Some nuclear imaging systems employ computed tomography (CT). CT is an imaging technique that uses x-rays to produce anatomical images. Magnetic Resonance Imaging (MRI/MR) is an imaging technique that uses magnetic fields and radio waves to generate anatomical and functional images. Some nuclear imaging systems combine images from PET and CT scanners during an image fusion process to produce images that show information from both a PET scan and a CT scan (e.g., PET/CT systems). For instance, CT scan data may be used to produce attenuation maps to correct PET scan data for attenuation. Similarly, some nuclear imaging systems combine images from PET and MRI scanners to produce images that show information from both a PET scan and an MRI scan.

In PET/CT imaging, low radiation dosing and lower exposure times are desirable for patient safety, comfortability and imaging volume throughput. Imaging dose in PET/CT comes from two sources: gamma radiation from injected PET isotopes and X-ray radiation from CT scan. CT data is used for attenuation and scatter corrections in PET image formation. A standard PET dose is typically needed to generate PET images of clinical quality so that physicians can make diagnoses with confidence. However, a standard PET dose and the combined CT exposure or MRI scan contributes to lower patient comfortability, longer scan times, and lower volume throughput. The techniques described in this disclosure can make CT or MR scans unnecessary for fully corrected activity image reconstruction. Another application of low-dose/count imaging with deep learning is that it may enable PET scanners with sparse detectors, which acquire less counts than normal PET scanners during the same time period since less detector blocks are used. Sparse detector configurations are sometimes desirable for cost saving. In this disclosure, low count and low-dose are used interchangeably.

The present disclosure is directed to overcoming this and other problems of the prior art.

### SUMMARY

In some embodiments, a computer-implemented method for image reconstruction is disclosed. The method includes receiving a low-dose PET image, applying a machine learning algorithm via a convolutional neural network to the low-dose PET image to generate an output image, wherein the output image includes correction for scatter and attenuation associated with the image being low-dose, and providing the output image to a computing device comprising a user interface.

In other embodiments, a computer-implemented method for training a neural network is disclosed. The method includes receiving standard-dose PET sinogram data comprising data points collected over a period of time, recreating low-dose PET sinogram data by selecting a subset of the standard-dose PET sinogram data, and reconstructing low-dose images based on the subset of the standard-dose PET sinogram data. The method also includes reconstructing standard-dose images based on the standard-dose PET sinogram data, correcting the standard-dose images for at least scatter and attenuation to produce corrected standard-dose images, and training a neural network based on the recreated low-dose images as input data and the corrected standard-dose images as target data.

In other embodiments, a system includes one or more memory devices storing a convolutional neural network, one or more interface devices, and at least one processor communicatively coupled to the one or more memory devices and one or more interface devices and configured to receive, by the one or more interface devices, a low-dose PET image, input the low-dose PET image to the convolutional neural network, and receive an output image from the convolutional neural network. The output image includes correction for scatter and attenuation associated with the image being low-dose and noise correction. The at least one processor is further configured to provide the output image to a display of the one or more interface devices.

In other embodiments, other computing devices and/or non-transitory computer readable mediums may store processing instructions for performing one or more steps associated with disclosed processes.

### BRIEF DESCRIPTION OF THE DRAWINGS

The following will be apparent from elements of the figures, which are provided for illustrative purposes and are not necessarily drawn to scale.

FIG. 1A illustrates a flow diagram of an exemplary image reconstruction process using a neural network, in accordance with some embodiments.

FIG. 1B illustrates a flow diagram of another exemplary image reconstruction process using a neural network, in accordance with some embodiments.

FIG. 2 illustrates a block diagram of an example computing device that can perform one or more of the functions described herein, in accordance with some embodiments.

FIG. 3 illustrates a flow diagram of an exemplary neural network training process, in accordance with some embodiments.

FIG. 4 illustrates an exemplary neural network, in accordance with some embodiments.

FIG. 5 illustrates a flowchart of an exemplary process for training a neural network, in accordance with some embodiments.

FIG. 6 illustrates a flowchart of an exemplary process for producing an image from low-dose PET data using a neural network, in accordance with some embodiments.

FIG. 7 illustrates the results of applying disclosed methods on brain imaging.

### DETAILED DESCRIPTION

This description of the exemplary embodiments is intended to be read in connection with the accompanying drawings, which are to be considered part of the entire written description.

The exemplary embodiments are described with respect to the claimed systems as well as with respect to the claimed methods. Furthermore, the exemplary embodiments are described with respect to methods and systems for image reconstruction, as well as with respect to methods and systems for training functions used for image reconstruction. Features, advantages, or alternative embodiments herein can be assigned to the other claimed objects and vice versa. For example, claims for the providing systems can be improved with features described or claimed in the context of the methods, and vice versa. In addition, the functional features of described or claimed methods are embodied by objective units of a providing system. Similarly, claims for methods and systems for training image reconstruction functions can be improved with features described or claimed in context of the methods and systems for image reconstruction, and vice versa.

Various embodiments of the present disclosure can employ machine learning methods or processes to provide clinical information from nuclear imaging systems. For example, the embodiments can employ machine learning methods or processes to reconstruct images based on captured measurement data, and provide the reconstructed images for clinical diagnosis. In some embodiments, machine learning methods or processes are trained, to improve the reconstruction of images, such as to simultaneously correct low-dose PET images for noise, scatter, and attenuation.

Low radiation dose is desirable in PET/CT imaging. The delivered dose originates from both CT scans and injected PET radioisotopes. CT data is used for attenuation and scatter corrections in PET image formation. A standard PET dose is usually needed to generate PET images of clinical quality so that physicians can make diagnosis with confidence. Disclosed embodiments may eliminate the CT scans and reduce the PET dose (i.e., in comparison to a standard does) while maintaining image quality by performing simultaneous attenuation correction, scatter correction, and de-noising using a deep learning approach. Low-dose PET scans can include different image data sets from different imaging conditions. For example, sinogram data sets associated with short scan duration, low contrast injection, low data counts, missing data, or other similar situations.

Disclosed embodiments include training of a multi-layer convolutional neural network (CNN) with non-attenuation corrected, non-scatter corrected, and low-dose PET images as input, and fully corrected standard-dose PET images as output (labels). After the CNN is trained, it may be used to generate fully corrected standard-dose equivalent PET images from low-dose PET data alone. This capability renders a CT/MR scan unnecessary and lowers a necessary PET dose significantly.

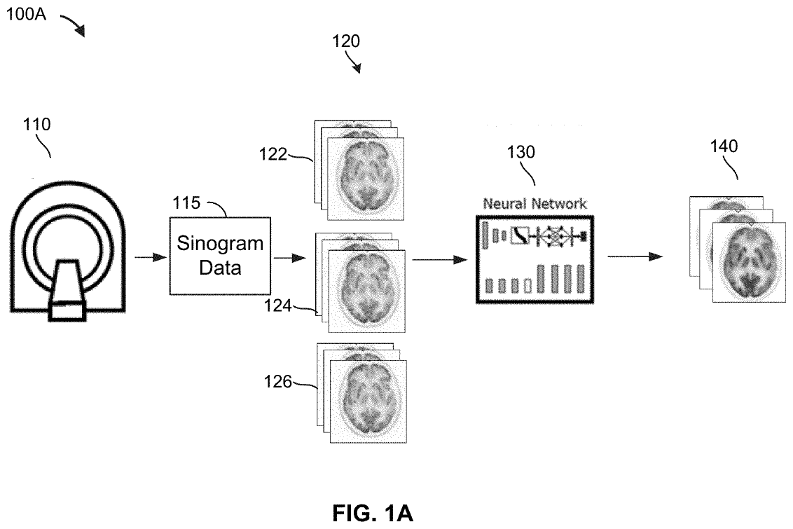

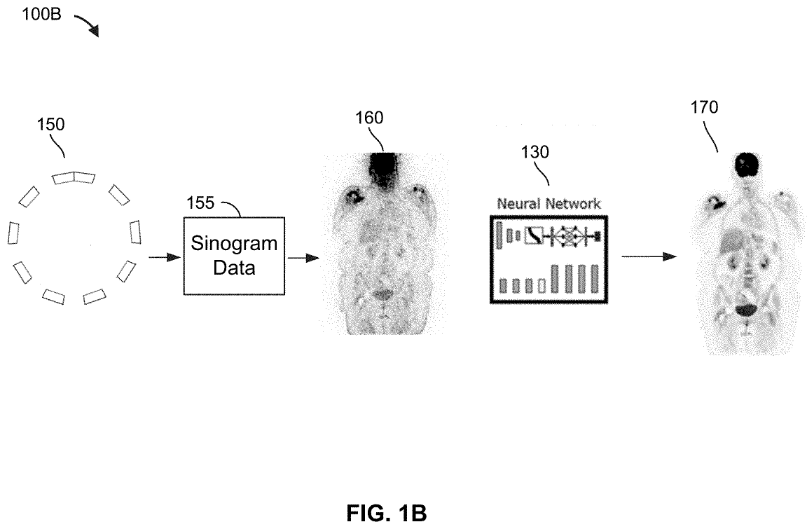

FIG. 1A illustrates one embodiment of a process flow 100A associated with an exemplary nuclear imaging system 110. In this example, a nuclear imaging system 100 employs an imaging pipeline using PET sinogram data 115 (e.g., time-of-flight (TOF) PET sinogram data). The imaging system 110 can perform one or more image reconstruction methods to produce images 120 from the sinogram data 115. According to disclosed embodiments, the imaging system 110 may input the images 120 to a neural network 130 to generate PET image volumes 140 having high quality for reliable clinical use.

In an exemplary embodiment, nuclear imaging system 110 includes an image scanning system and image reconstruction system. The image scanning system can be, for example, a PET/CT scanner or a MR/PET scanner. The image scanning system generates sinogram data 115, such as TOF sinograms. The sinogram data 115 can represent anything imaged in the scanner's field-of-view (FOV) containing positron emitting isotopes. For example, the sinogram data 115 can represent whole-body image scans, such as image scans from a patient's head to thigh. In some examples, all or parts of image reconstruction system are implemented in hardware, such as in one or more field-programmable gate arrays (FPGAs), one or more application-specific integrated circuits (ASICs), one or more state machines, digital circuitry, or any other suitable circuitry. In some examples, parts or all of the image reconstruction system can be implemented in software as executable instructions such that, when executed by one or more processors, cause the one or more processors to perform respective functions as described herein. The instructions can be stored in a non-transitory, computer-readable storage medium, for example.

In an exemplary embodiment, the sinogram data 115 includes data associated with low-dose PET scans. A low-dose PET scan may include image data associated with a shorter scan duration, less contrast injection (and therefore fewer events to detect), fewer data counts (regardless of scan duration), or other data sets that may result in lower image quality as compared to a full data set from a standard-dose PET scan. For instance, a standard-dose PET scan may occur over 900 seconds, while a low-dose PET scan may occur over 90 seconds. The sinogram data 115 from a low-dose PET scan may be transformed into the low-dose images 120. The low-dose images 120 can be, for example, low-dose un-attenuation corrected images 122, such as images reproduced based on sinogram data 115, without correction for attenuation that may conventionally occur based on a corresponding CT result. Accordingly, disclosed embodiments may be associated with generating fully-corrected images based on low-dose PET scan data without the need for CT scan data. The images 120 may additionally or alternatively include partially-attenuation corrected PET images 124, such as images associated with some attenuation correction after a low-dose CT scan (e.g., short-duration CT scan). The images 120 may additionally or alternatively include low-dose activity images 126. The images 120, including one or more of the un-attenuation corrected images 122, partially attenuation corrected images 124, or low-dose activity images 126, can be generated by the imaging system 110 using an approximation algorithm, such as an ordinary Poisson ordered subsets expectation maximization (OP-OSEM) algorithm or a Maximum Likelihood Attenuation and Activity (MLAA) estimation.

According to disclosed embodiments, one or more images 120 associated with low-dose PET scans (e.g., as described above in relation to example images 122, 124, 126) are input to the neural network 130 to provide image corrections that may otherwise occur based on available scan data (e.g., from a standard-dose PET and CT scan). The neural network 130 may simultaneously correct for noise, scatter, and attenuation to produce standard-dose, fully corrected PET images 140, which can be a multi-slice image volume. Final images 140 can include image data that can be provided for display and analysis.

FIG. 1B illustrate another embodiment of a process flow 100B. The process flow 100B is associated with a nuclear imaging system 150 having sparse detectors. Sparse detector configurations in a PET scanner may be desirable to save costs. The nuclear imaging system 150 having sparse detectors may complete a PET scan to collect sinogram data 155. The sinogram data 155 may be considered “low-dose” data in that it may include a low count (i.e., smaller data set compared to a standard-dose PET scan performed on a normal PET scanner). Uncorrected reconstruction is then performed on the low count sinogram data 155 to obtain a low count uncorrected image 160. The low-count uncorrected image 160 can be input to the neural network 130 to produce a standard-dose, fully-corrected image 170.

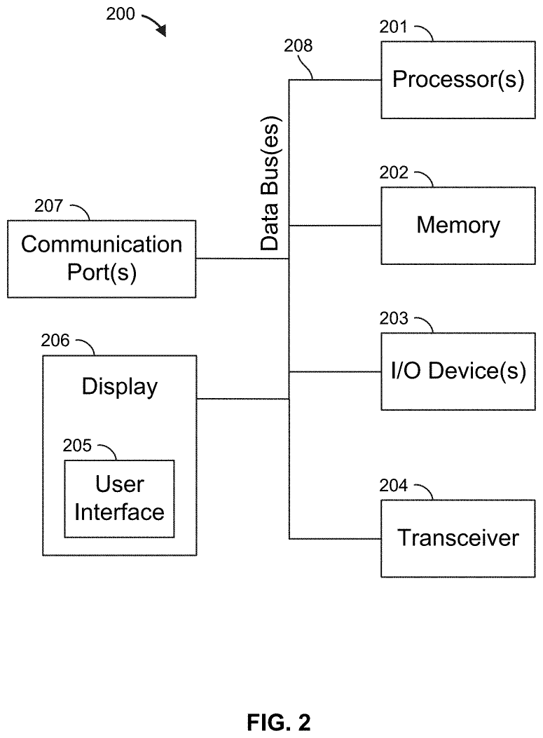

FIG. 2 illustrates a computing device 200 that can be employed by an imaging system, such as nuclear imaging system 110. Computing device 200 can implement, for example, one or more of the functions described herein. For example, computing device 200 can implement one or more of the functions of an imaging system, such as image reconstruction processes related to data gathered by the nuclear imaging system 110. In some embodiments, the computing device 200 may represent computing components associated with the neural network 130.

Computing device 200 can include one or more processors 201, memory 202, one or more input/output devices 203, a transceiver 204, one or more communication ports 207, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Processors 201 can be configured to perform a certain function or operation by executing code, stored on instruction memory 207, embodying the function or operation. For example, processors 201 can be configured to perform one or more of any function, method, or operation disclosed herein.

Memory 202 can include an instruction memory that can store instructions that can be accessed (e.g., read) and executed by processors 201. For example, the instruction memory can be a non-transitory, computer-readable storage medium such as a read-only memory (ROM), an electrically erasable programmable read-only memory (EEPROM), flash memory, a removable disk, CD-ROM, any non-volatile memory, or any other suitable memory. For example, the instruction memory can store instructions that, when executed by one or more processors 201, cause one or more processors 201 to perform one or more of the functions of an image reconstruction system.

Memory 202 can also include a working memory. Processors 201 can store data to, and read data from, the working memory. For example, processors 201 can store a working set of instructions to the working memory, such as instructions loaded from the instruction memory. Processors 201 can also use the working memory to store dynamic data created during the operation of computing device 200. The working memory can be a random access memory (RAM) such as a static random access memory (SRAM) or dynamic random access memory (DRAM), or any other suitable memory.

Input-output devices 203 can include any suitable device that allows for data input or output. For example, input-output devices 203 can include one or more of a keyboard, a touchpad, a mouse, a stylus, a touchscreen, a physical button, a speaker, a microphone, or any other suitable input or output device.

Communication port(s) 207 can include, for example, a serial port such as a universal asynchronous receiver/transmitter (UART) connection, a Universal Serial Bus (USB) connection, or any other suitable communication port or connection. In some examples, communication port(s) 207 allows for the programming of executable instructions in instruction memory 207. In some examples, communication port(s) 207 allow for the transfer (e.g., uploading or downloading) of data, such as sinograms (e.g., sinogram data 115).

Display 206 can display user interface 205. User interfaces 205 can enable user interaction with computing device 200. For example, user interface 205 can be a user interface for an application that allows for the viewing of final images generated by an imaging system. In some examples, a user can interact with user interface 205 by engaging input-output devices 203. In some examples, display 206 can be a touchscreen, where user interface 205 is displayed on the touchscreen.

Transceiver 204 can allow for communication with a network, such as a Wi-Fi network, an Ethernet network, a cellular network, or any other suitable communication network. For example, if operating in a cellular network, transceiver 204 is configured to allow communications with the cellular network. Processor(s) 201 is operable to receive data from, or send data to, a network via transceiver 204.

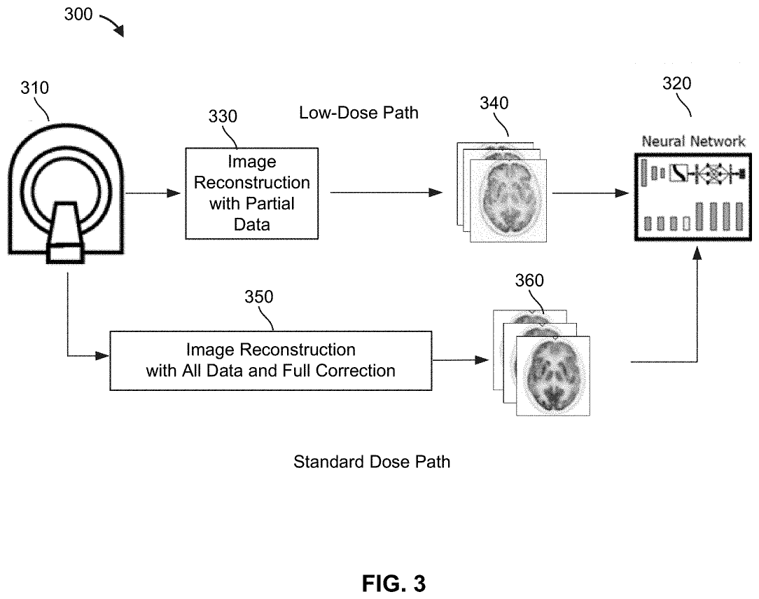

FIG. 3 illustrates a diagram of process 300 for using PET imaging data from a nuclear imaging system 310 to train a neural network 320 to simultaneously perform image correction for noise, scatter, and attenuation. The nuclear imaging system 310 may be a combined PET/CT system or other similar system for collecting standard-dose imaging data, such as PET and MR data. The process 300 may include a low-dose path for generating input data for training the neural network 320 and a standard-dose path for generating fully-corrected images as targets or labels associated with the input data. According to some disclosed embodiments, the neural network 320 is a deep learning convolutional neural network.

In the process 300, the nuclear imaging system 310 produces standard-dose PET image data, such as sinogram data associated with a typical complete scan (e.g., approximately 900 seconds of scan data). In the low-dose path, the nuclear imaging system 310 performs image reconstruction 330 using only a portion of the data that may be used to represent a low-dose scan. For instance, the nuclear imaging system 310 may use only 90 seconds of scan data to produce images 340 (e.g., sinograms). By using only a portion of the standard-dose PET data, the nuclear imaging system 310 may recreate or mimic low-dose images. The images 340 are input to the neural network 320 as input data sets for training the neural network 320. The images 340 are not corrected for noise, scatter, or attenuation, and thus may be blurry, low-count, and/or unreliable for diagnostic use. In some embodiments, the image reconstruction 330 and images 340 may be associated with actual low-dose imaging data (e.g., a PET scan of only 90 seconds in duration instead of a selected portion of a standard PET scan).

In the standard-dose path, the nuclear imaging system 310 may perform a standard image reconstruction 350 using all of the collected data (e.g., 900 seconds of PET scan data) to produce fully corrected images 360, which are de-noised, attenuation, and scatter corrected. The fully corrected images 360 are provided to the neural network 320 and associated with the image 340 as “target” (sometimes referred to as “labels”) images to train the neural network 320.

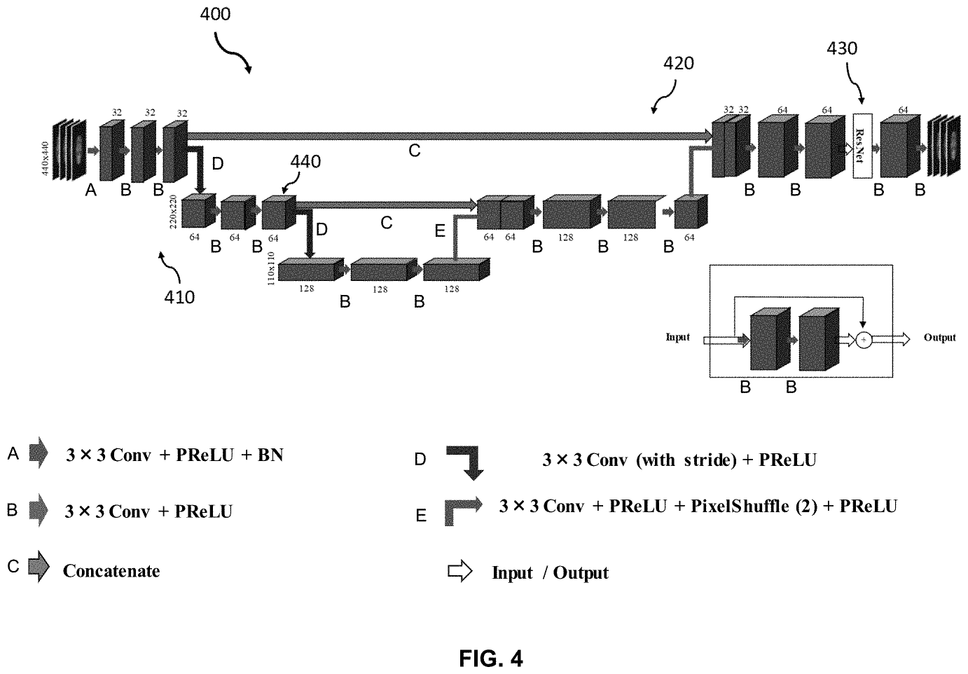

FIGS. 4 is a diagram of an exemplary convolutional neural network 400 that may be trained to perform the simultaneous corrections described herein. The neural network 400 is exemplary and other neural network architectures and configurations can be used for training and image processing. In the displayed embodiment, the convolutional neural network 400 has a modified U-net architecture. In FIG. 4, cony stands for convolution, BN stands for batch normalization, and PReLU stands for parameterized rectified linear unit. The neural network 400 can include a down-sampling phase 410 and an up-sampling phase 420. The down sampling phase 410 can take four slices of an input image, then apply a sequence of 3×3 convolutional layers, PReLU layers, BN layer, and 3×3 convolutional layers with stride 2 (down-sampling). The up-sampling phase 420 can continuously apply 3×3 convolutional layers, PReLU layers, and PixelShuffle layers with an upscale factor of 2 (up-sampling). The output of the up-sampling phase can be input to a ResNet 430 to generate four slices of the output image. Each box corresponds to a multi-channel feature map. The number of channels in each feature map is indicated above or below the boxes. At each down sample or up-sample step, the feature map size is halved or doubled. For example, after first down sample step, the size of feature map is changed from 440×440 to 220×220. Each up-sampled output can be concatenated to its counterpart's output on the left to re-capture the information in earlier layers. With proper padding, the output of the neural network 400 maintains the same size as the input. A loss function of the neural network 400 can combine, in one embodiment, a weighted Mean Absolute Error (MAE), a Multiscale Structural Similarity (MS-SSIM) loss, and a content loss with VGG19. The weights of each loss component can be dynamically adjusted in some embodiments. It should be noted that other variants of deep convoluted neural network can be designed to achieve similar task and the parameters related to networks can be changed. For example, the number of input image slices can be 159 and the size of the convolution kernel can be 3×3×3.

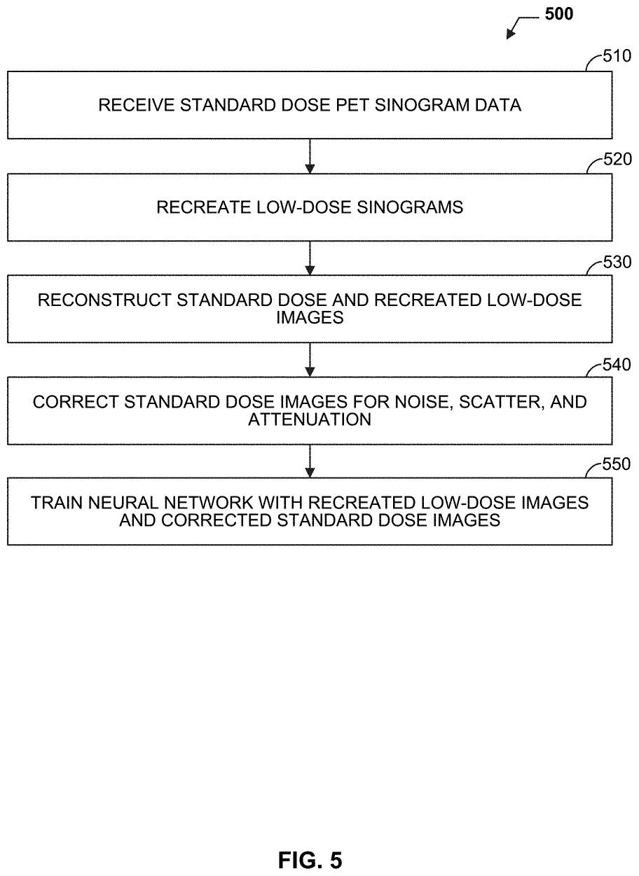

FIG. 5 is a flowchart of an exemplary process 500 for training and using a neural network in accordance with disclosed embodiments. One or more processors (e.g., processor 201) may be configured to execute software instructions to perform one or more steps of the process 500.

In step 510, the processor receives standard-dose PET sinogram data. For example, a nuclear imaging system may perform a scan of a patient using a standard-dose of radiation (e.g., exposure time, contrast amount, etc.) according to conventional methods. The standard-dose PET data may include CT and/or MR data simultaneously and/or separately acquired. While sinogram data is described, it should be understood that data formats may vary (e.g., listmode data, binned data, etc.).

In step 520, the processor recreates low-dose sinogram data sets. For instance, the processor may select a subset of data from the standard-dose PET sinogram data (e.g., a 10% selection of the full data count, or other subset amount depending on the application). The subset of selected data may represent a low-dose dataset in that a low-dose dataset typically includes a shorter scan duration and thus lower counts of data points. The processor may also select a subset of data from sinogram data acquired on a normal PET scanner to mimic low count data acquired on a PET system with sparse detector configurations. The low-dose multi-slice images may comprise an axial depth of 4, for example.

In step 530, the processor reconstructs standard-dose and recreated low-dose images. For instance, the processor may separately produce activity images associated with the full or complete data set (i.e., the standard-dose sinograms) and with the subset of data (i.e., the recreated low-dose sinograms). The standard-dose sinograms include more data points (counts) and thus may include higher quality and granularity images. However, both image sets may suffer from typical sinogram approximation drawbacks, such as noise, scatter, and attenuation.

The processor may be configured to use scanner-specific normalization measures that include various components (for example, crystal efficiency, crystal interference pattern, dead time correction parameters, etc.) for adjusting the PET raw data. The processor may perform un-corrected (no attenuation and no scatter corrections) image reconstruction with an OP-OSEM algorithm using the low-dose/count raw emission sinogram data and normalization components which are expanded into sinogram format.

In step 540, the processor corrects the standard-dose activity images for noise, scatter, and attenuation. For example, the processor may use conventional methods known for correcting sinogram reconstructions, such as applying corrections based on attenuation maps generated based on CT or MR scan data.

In step 550, the processor trains a neural network with the recreated low-dose images and the corrected standard-dose images. For example, the low-dose PET images may be used as training input and the fully corrected standard dose PET images may be the target data (e.g., “label” or “ground truth” of the neural network training). In training the neural network, the processor may implement a loss function for quantifying an error associated with a combination of noise, scatter, and attenuation and train the neural network to minimize the loss function. For example, the loss function of the neural network may be a combination of mean absolute error and multi-structural similarity loss. The neural network may be trained to measure an error via the loss function and compare the error to a threshold.

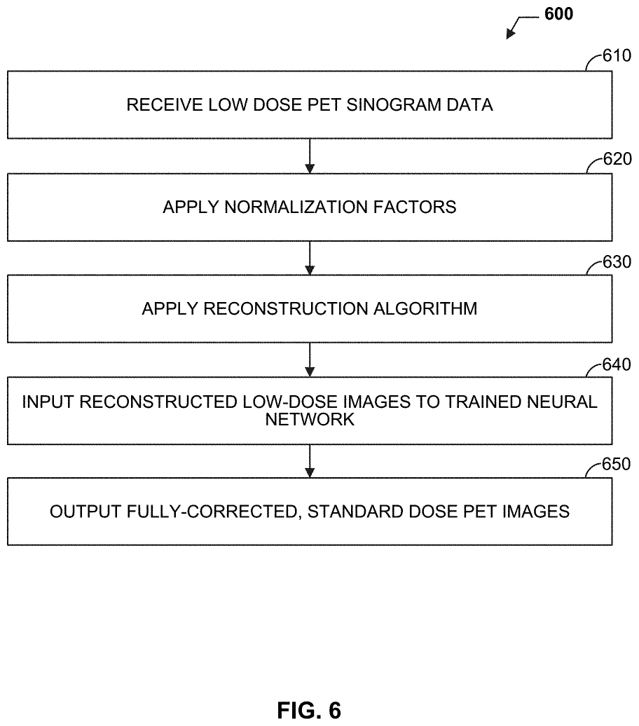

FIG. 6 is a flowchart of an exemplary process 600 to use a neural network to perform image correction of low-dose PET images, such as using a neural network trained in process 500. One or more processors (e.g., processor 201) may be configured to execute software instructions to perform one or more steps of the process 600.

In step 610, the processor receives low-dose PET sinogram data. The low-dose PET sinogram data may be associated with a PET scan that occurs with less-than-conventional radiation exposure (e.g., via exposure time, contrast amount, etc.) or on a system with sparse detector configurations. In another example, the low-dose PET sinogram data may be associated with some other cause of a low-count data set, such as a sparse detector configuration of an imaging system. In step 620, the processor may apply normalization factors to adjust the sinogram data for scanner-specific features. The processor may also perform data filtering, such as subtracting randoms from the data set.

In step 630, the processor applies a reconstruction algorithm to the normalized low-dose PET sinogram data. For instance, the processor may apply an OP-OSEM algorithm to produce a low-dose PET image, although other reconstruction algorithms are possible, such as an MLAA estimation. Each of the following may be considered a low-dose PET image: low-dose un-attenuation corrected PET images, low-dose partially attenuation corrected PET images, and low-dose activity images generated from an MLAA estimation. Accordingly, the PET data may be collected without measured attenuation data or with only partially measured attenuation data, thereby removing the requirement for accompanying CT data or otherwise reducing a scan duration for acquiring such CT data (in the example of only partially corrected attenuation).

In some embodiments, partial correction of attenuation may be associated with a partial CT scan captured during the PET scan. The partial CT may be used to reduce a radiation dose in a long PET scanner. For example, a PET scanner with long axial field of view (FOV) is able to cover the whole torso. However, a CT scan may be only performed over the chest region. With only partial CT data, it is difficult to perform a fully-corrected reconstruction using all PET data. However, the partial CT data can be used for a partial attenuation correction to produce partially-corrected images for input to the neural network.

In step 640, the processor may provide the reconstructed low-dose images to the trained neural network for simultaneous correction of scatter, attenuation, and noise associated with the low-dose images. The neural network may be stored in a memory (e.g., one or more memory devices) in communication with the processor. In some embodiments, the processor inputting the images to the neural network may be separated from a dedicated neural network processor. The neural network processor may receive the input images and perform an image transformation process to output a fully-corrected image comparable to such images that may have been generated from a standard dose image reconstruction process. In step 650, the processor may output the fully-corrected images, such as by displaying them to a user for analysis and/or diagnostic review.

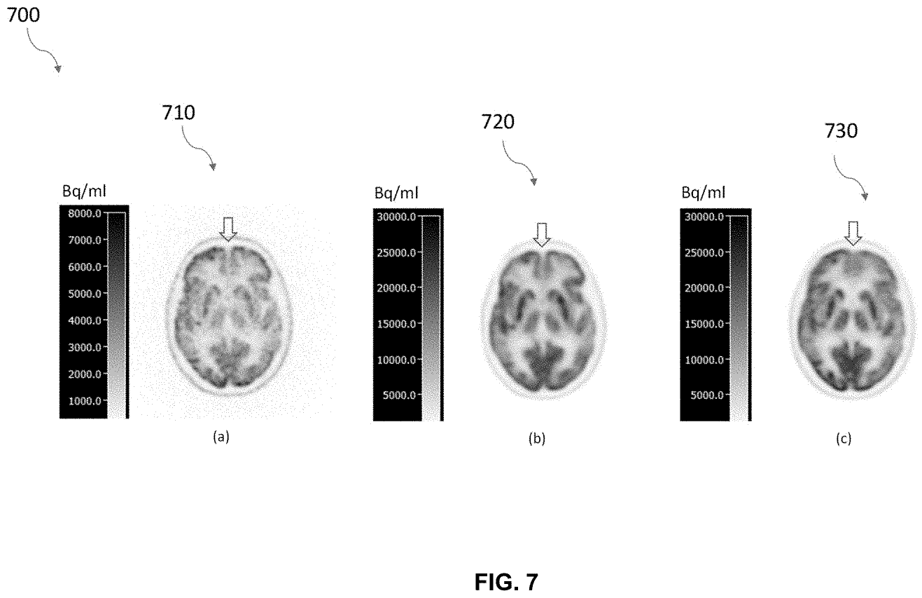

FIG. 7 includes example images illustrating the results of applying disclosed methods on brain imaging. In one example, low-count sinogram data associated with the first 90seconds of listmode data from a 900-second scan is obtained along with the full 900-second data set. The image 710 shows an un-corrected image reconstructed from the low-count sinogram data (i.e., using the first 90 seconds of listmode data). The image 720 shows the output image of an exemplary trained deep CNN 710 as input. The image 730 shows a fully-corrected image reconstructed from the full 900-second data set using the standard OSEM algorithm. The background activity outside of the skull in the image 710 is due to uncorrected scatter. The suppressed reconstructed value towards the center of the image 710 is due to uncorrected attenuation. Compared to 710, the image 720 is fully corrected for both attenuation and scatters and its noise level is similar to the image 730, which is reconstructed from the full 900 seconds of data with all corrections using OP-OSEM algorithm.

The disclosed embodiments provide training of a neural network to provide simultaneous corrections for various imaging discrepancies when low-dose image reconstructions are input. The disclosed processes may be tailored to make some corrections to training data (e.g., by applying normalization factors and subtracting randoms) such that the neural network is trained to particular corrections, such as attenuation, scatter, and/or noise.

In one example, a multi-layer convolutional neural network may be trained to convert non-attenuation and non-scatter corrected low count PET images directly to fully corrected high count PET images. The disclosed embodiments thus provide an ability to generate standard diagnostic PET images without CT or MR scan from low-dose PET data. The disclosed embodiments are particularly applicable to situations that require or desire a minimal radiation dose, such as in pediatric PET neuroimaging where very low radiation exposure is paramount.

The disclosed embodiments include a neural network trained to implement a loss function that encompasses multiple imaging errors, at least including attenuation and scatter and in some embodiments also including noise. A convolutional neural network trained to include such a loss function may operate in iteration across layers to eventually produce a fully-corrected image determined based on a comparison of a result of the loss function to a threshold. Thus, the disclosed embodiments provide a simultaneous correction of multiple errors or causes of low image quality, thereby enabling the low-dose input described herein and the associated low-exposure and patient comfortability advantages already described.

The apparatuses and processes are not limited to the specific embodiments described herein. In addition, components of each apparatus and each process can be practiced independent and separate from other components and processes described herein.

The previous description of embodiments is provided to enable any person skilled in the art to practice the disclosure. The various modifications to these embodiments will be readily apparent to those skilled in the art, and the generic principles defined herein can be applied to other embodiments without the use of inventive faculty. The present disclosure is not intended to be limited to the embodiments shown herein, but is to be accorded the widest scope consistent with the principles and novel features disclosed herein.

What is claimed is:

1. A computer-implemented method, comprising:
receiving a low-dose PET image;
applying a machine learning algorithm via a convolutional neural network to the low-dose PET image to generate an output image, wherein the output image includes correction for scatter and attenuation associated with the image being low-dose; and
providing the output image to a computing device comprising a user interface.

2. The computer-implemented method of claim 1, wherein the low-dose PET image is reconstructed from low-dose PET data using an OP-OSEM algorithm.

3. The computer-implemented method of claim 2, wherein the low-dose PET data is the result of a scan duration of equal to or less than 90 seconds.

4. The computer-implemented method of claim 2, wherein the low-dose PET data is associated with a sparse detector configuration of a PET scanner.

5. The computer-implemented method of claim 2, wherein the low-dose PET data is corrected for scanner-specific normalization factors.

6. The computer-implemented method of claim 1, wherein the low-dose PET image is not corrected for attenuation.

7. The computer-implemented method of claim 1, wherein the low-dose PET image is partially corrected for attenuation.

8. The computer-implemented method of claim 1, wherein the low-dose PET image is an activity image reconstructed from a maximum likelihood activity and attenuation estimation.

9. A computer-implemented method for training a neural network, comprising:
receiving standard-dose PET sinogram data comprising data points collected over a period of time;
recreating low-dose PET sinogram data by selecting a subset of the standard-dose PET sinogram data;
reconstructing low-dose images based on the subset of the standard-dose PET sinogram data;
reconstructing standard-dose images based on the standard-dose PET sinogram data;
correcting the standard-dose images for at least scatter and attenuation to produce corrected standard-dose images; and
training a neural network based on the recreated low-dose images as input data and the corrected standard-dose images as target data.

10. The computer-implemented method of claim 9, wherein reconstructing the low-dose images comprises using an OP-OSEM algorithm.

11. The computer-implemented method of claim 9, wherein reconstructing the low-dose images comprises using a maximum likelihood of activity and attenuation estimation.

12. The computer-implemented method of claim 9, wherein the subset of the standard-dose PET sinogram data includes data collected over a subset of the period of time.

13. The computer-implemented method of claim 12, wherein the subset of the period of time is approximately 10%-50% of the period of time.

14. The computer-implemented method of claim 9, further comprising correcting the standard-dose images for noise.

15. The computer-implemented method of claim 9, wherein the neural network is a multi-layer convolutional neural network.

16. A system, comprising:
one or more memory devices storing a convolutional neural network;
one or more interface devices; and
at least one processor communicatively coupled to the one or more memory devices and one or more interface devices and configured to:
receive, by the one or more interface devices, a low-dose PET image;
input the low-dose PET image to the convolutional neural network;
receive an output image from the convolutional neural network, wherein the output image includes correction for scatter and attenuation associated with the image being low-dose and noise correction; and
provide the output image to a display of the one or more interface devices.

17. The system of claim 16, wherein the neural network is configured to perform the correction for scatter, attenuation, and noise simultaneously.

18. The system of claim 16, wherein the at least one processor is further configured to generate the low-dose PET image using an OP-OSEM algorithm.

19. The system of claim 16, wherein the low-dose PET image is not corrected for attenuation.

20. The system of claim 16, wherein the low-dose PET image is partially corrected for attenuation.
